(19) United States
(12) Patent Application Publication
(10) Pub. No.: US 2013/0072790 A1
(43) Pub. Date: Mar. 21, 2013

(54) SELECTION AND OPTIMIZATION FOR CARDIAC RESYNCHRONIZATION THERAPY

(71) Applicants: Daniel Ryder Ludwig, Maple Glen, PA (US); Mati Friehling, Pittsburgh, PA (US); David Schwartzman, Pittsburgh, PA (US)

(72) Inventors: Daniel Ryder Ludwig, Maple Glen, PA (US); Mati Friehling, Pittsburgh, PA (US); David Schwartzman, Pittsburgh, PA (US)

(73) Assignee: University of Pittsburgh-Of the Commonwealth System of Higher Education, Pittsburgh, PA (US)

(21) Appl. No.: 13/621,962

(22) Filed: Sep. 18, 2012

Related U.S. Application Data
(60) Provisional application No. 61/536,310, filed on Sep. 19, 2011.

(51) Int. Cl. A61B 6/00; G06F 17/00

(52) U.S. Cl. CPC A61B 6/52; G06F 17/00
USPC 600/425; 703/11

(57) ABSTRACT

Systems and methods for imaging and analyzing a patient heart are described. Imaging can be performed with a variety or combination of methods, including single photon emission computed tomography, to provide a comprehensive, three-dimensional image or model of the heart including high-resolution details relating to scar tissue and other abnormalities. Data, including information related to the heart developed through the imaging process, can be analyzed to determine if a patient is a desirable candidate for cardiac resynchronization therapy. Specific details relating to a cardiac resynchronization therapy device and a procedure for implantation can be developed through analysis of available information. A heart model and “virtual roadmap” can be generated to guide a medical practitioner through patient-individualized procedures related to the specific details gleaned through analysis.

### CROSS-REFERENCE TO RELATED APPLICATIONS

This application is related to U.S. Provisional Patent Application Ser. No. 61/536,310 entitled “A SYSTEM FOR SELECTION AND OPTIMIZATION OF PATIENTS UNDERGOING CARDIAC RESYNCHRONIZATION THERAPY” and filed Sep. 19, 2011, and claims the benefit thereof. The entirety of the above-noted application is incorporated by reference herein.

### BACKGROUND

This disclosure relates generally to medical procedures, and more particularly, toward imaging and analyzing patient hearts to optimize cardiac resynchronization therapy outcomes.

Heart failure (HF) is a major cause of morbidity and mortality, and can result in high medical costs to patients who have had heart failure or are at high risk for heart failure. The principal cause of heart failure in the western world is prior myocardial infarction (MI), which results from coronary artery obstruction causing formation of a scar replacing healthy heart muscle. Scar decreases the vigor of heart contraction. In some patients, the MI also damages the wiring of the remaining healthy muscle. The resulting asynchrony (or dyssynchrony) of this muscle causes a further diminishment in contraction vigor.

In recent years, pacemaker systems have been developed which have the potential to remediate asynchrony. Implantation of such systems is termed cardiac resynchronization therapy (CRT). It turns out that CRT does not benefit a substantial minority of heart failure patients, because their hearts lack appropriate levels of cardiac asynchrony. It is important to identify this subset of patients in advance, because delivery of CRT carries risk and is and costly, and may not be pursued if the likelihood of the sought benefits is low. Further, among patients who do respond to CRT, it turns out that the way the pacemaker system is configured has an important impact on the magnitude of the response.

Common techniques for assessing patient heart condition in this context suffer from a variety of limitations. Many cardiac diagnostic tests fail to provide the necessary detail and volume of information necessary to perform confident analysis. While cardiac magnetic resonance imaging alone can yield data of useful resolution, many patients cannot undergo contrast-enhanced magnetic resonance imaging due to the presence of existing pacemakers and/or renal dysfunction. In another example, echocardiography alone can result in unacceptably low scar detection capability.

Accordingly, there is a need to better identify patients who are likely to be responsive to cardiac resynchronization therapy, and to optimize both the device and its implantation in conjunction with the therapy.

### SUMMARY

The following presents a simplified summary in order to provide a basic understanding of some aspects of the disclosed aspects. This summary is not an extensive overview and is intended to neither identify key or critical elements nor delineate the scope of such aspects. Its purpose is to present some concepts of the described features in a simplified form as a prelude to the more detailed description that is presented later.

The innovation disclosed and claimed herein, in one aspect thereof, comprises a use of single proton emission computed tomography (SPECT) as a basis for CRT patient selection. Previous attempts to provide similar information utilized echocardiography or magnetic resonance imaging modalities. Each of these imaging modalities can encounter limitations in comparison to single proton emission computed tomography. Further, SPECT imaging has minimal risk as it is painless, non-invasive, non-toxic, widely available, relatively inexpensive, and associated with automated systems.

In accordance with one or more aspects and corresponding disclosure thereof, various aspects are described in connection with single proton emission computed tomography imaging techniques for predicting success and optimizing cardiac resynchronization therapy devices and procedures.

In an aspect of the subject innovation, software and circuit logic and algorithms can be employed to identify patients with a high likelihood of benefit from cardiac resynchronization therapy. Analysis can be performed on heart images to render this determination based on imaged, detected, and/or sensed characteristics relating to the patient's heart.

In another aspect of the subject innovation, an “individualized prescription” for heart treatment can be developed. Computerized or circuit logic can be employed to analyze aspects of the patient to specify or modify aspects of a general treatment procedure. For example, specific details relating to a cardiac resynchronization device and surgery for implanting the same can be developed based on the particulars and peculiarities of an individual patient heart.

In another aspect of the subject innovation, a multi-dimensional model of a patient's heart can be generated for further analysis and/or to guide a medical practitioner in conducting treatment to the heart and/or patient. In an example, a surgeon can utilize a patient-specific 3-dimensional heart model in conjunction with the planning for and implantation of a cardiac resynchronization therapy device.

To the accomplishment of the foregoing and related ends, one or more aspects comprise the features hereinafter fully described and particularly pointed out in the claims. The following description and the annexed drawings set forth in detail certain illustrative aspects and are indicative of but a few of the various ways in which the principles of the aspects may be employed. Other advantages and novel features will become apparent from the following detailed description when considered in conjunction with the drawings and the disclosed aspects are intended to include all such aspects and their equivalents.

### BRIEF DESCRIPTION OF THE DRAWINGS

FIG. 1 illustrates a block diagram of an example system for imaging and analyzing a portion of a patient.

FIG. 2 illustrates a block diagram of an example system for generating a model of a portion of a patient.

FIG. 3 illustrates a block diagram of an example system for imaging and analyzing a patient heart, as well as generating a model for use in treating the patient.

FIG. 4 illustrates a block diagram of an example system for providing viability and optimization information related to a heart.

FIG. 5 illustrates a flow diagram of an example methodology for selecting and treating patients based on imaging information related to the patient's heart.

FIG. 6 illustrates a flow diagram of an example methodology for imaging a patient heart.

FIG. 7 illustrates a flow diagram of an example methodology for generating a model of a patient heart.

FIG. 8 illustrates a brief general description of a suitable computing environment wherein the various aspects of the subject innovation can be implemented.

FIG. 9 illustrates a schematic diagram of a client—server-computing environment wherein the various aspects of the subject innovation can be implemented.

### DETAILED DESCRIPTION

The innovation disclosed and claimed herein, in one aspect thereof, comprises a use of single proton emission computed tomography as a basis for cardiac resynchronization therapy patient selection. Upon selection, individually-developed devices and implantation plans can be developed and implanted with a series of algorithms such as those in the proprietary SmartPace™ software. Other aspects will be appreciated by those skilled in the art upon study of the disclosures herein.

In some embodiments, image viewing and analysis can be performed in accordance with disclosures herein using the freely available software Segment, available at http://segment.heiberg.se. The Segment software can be used alone or in combination with other platforms, plug-ins or modules (e.g., programming environments or user interfaces for the development of algorithms, analysis of information, and complex computation) without departing from the scope of the subject innovation.

As used herein, the word “prescription” is intended to relate to medical procedures in general. Disregarding colloquial suggestion, a “prescription” need not have any relation to drugs, antibiotics, or other orally or intravenously applied substances. While a prescription can include such aspects, as used herein, a prescription can also relate to, for example, a particular means of completing any medical procedure. In a specific example, a prescription to implant a cardiac resynchronization therapy device can include details on the configuration, construction and/or settings of the device, as well as a “virtual roadmap” (or step-by-step custom instructions) particularized to the patient, based at least on the presence of scarring in the heart and images of the heart. Other aspects related to prescriptions will become apparent in view of the disclosures herein, and the above is intended to ensure understanding of a sufficiently broad, flexible use of the term “prescription,” rather than any particular limiting embodiment, interpretation or construction.

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable or script, a thread of execution, a program, a computer, and/or information relevant to effecting the desired function. By way of illustration, both an application running on a server and the server can be a component. One or more components may reside within a process and/or thread of execution and a component may be localized on one computer and/or distributed between two or more computers.

The word “exemplary” is used herein to mean serving as an example, instance, or illustration. Any aspect or design described herein as “exemplary” is not necessarily to be construed as preferred or advantageous over other aspects or designs.

Furthermore, the one or more versions may be implemented as a method, apparatus, or article of manufacture using standard programming and/or engineering techniques to produce software, firmware, hardware, or any combination thereof to control a computer to implement the disclosed aspects. The term “article of manufacture” (or alternatively, “computer program product”) as used herein is intended to encompass a computer program accessible from any computer-readable device, carrier, or media. For example, computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope of the disclosed aspects.

Various aspects will be presented in terms of systems that may include a number of components, modules, and the like. It is to be understood and appreciated that the various systems may include additional components, modules, et cetera and/or may not include all of the components, modules, et cetera discussed in connection with the figures. A combination of these approaches may also be used. The various aspects disclosed herein can be performed on electrical devices including devices that utilize touch screen display technologies and/or mouse-and-keyboard type interfaces. Examples of such devices include computers (desktop and mobile), smart phones, personal digital assistants (PDAs), and other electronic devices both wired and wireless.

Various aspects are now described with reference to the drawings. In the following description, for purposes of explanation, numerous specific details are set forth in order to provide a thorough understanding of one or more aspects. It may be evident, however, that the various aspects may be practiced without these specific details. In other instances, well-known structures and devices are shown in block diagram form in order to facilitate describing these aspects.

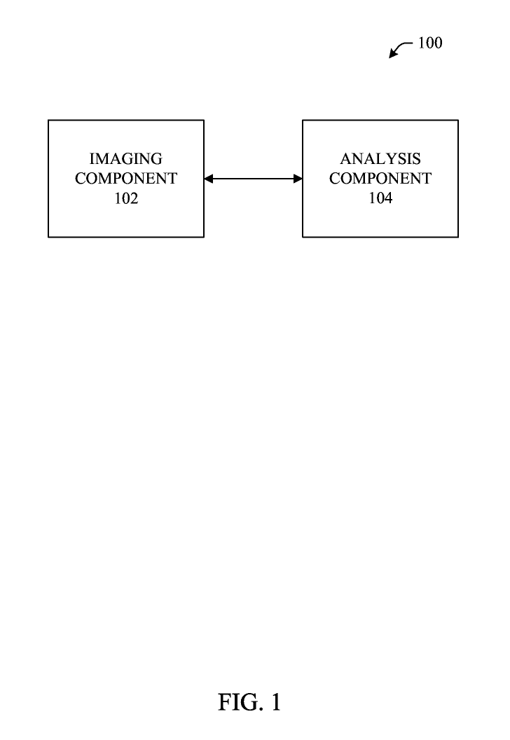

FIG. 1 illustrates example system 100 including imaging component 102 and analysis component 104. Imaging component 102 can include or receive information from equipment configured to render images of a live human heart. Analysis component 104 can include means for analyzing the heart images.

Imaging component 102 can include, or receive information from, equipment effecting, for example, single-photon emission computed tomography applied to or around a heart. In some embodiments, other nuclear medicine techniques can be employed alternatively or in combination to facilitate localized imaging of a heart. Single-photon emission computed tomography can employ, in some embodiments, gamma rays to facilitate imaging through multiple layers of tissue to generate a true three-dimensional representation of the heart and adjacent tissue. In some embodiments, alternative techniques employing other tissue-penetrating energy or fields (e.g., x-ray, sonography, infrared light, magnetic imaging, electrocardiography), particles or anti-particles (positron emission imaging techniques), internal diagnostic equipment (e.g., intravascular micro-devices), and combined techniques (e.g., magnetic resonance imaging including contrast dye) can be employed to develop information useful to imaging component 102. Various cameras can be employed to gather information related to the patient. In some embodiments (e.g., electrocardiography), imaging component 102 can collect or utilize data that does not correspond to a literal captured image (e.g., electrical activity) for standalone use or use in conjunction with captured images.

In some embodiments, a gated SPECT technique can be employed. Gated SPECT techniques can include SPECT procedures where an electrocardiogram guides acquisition such that the resulting SPECT images show heart contraction over an interval measured between “R waves.” An R wave can be represented as a prominent spike in an electrocardiogram schematic that occurs (in conjunction with other wave activity) during depolarization of the right and left ventricles. In embodiments, gated SPECT techniques can also select other periods to observe within the heart rhythms, and/or multiple contractions in the heart.

Specific techniques operable with embodiments can include phase analysis of gated SPECT myocardial perfusion imaging (MPI). Phase analysis of gated SPECT MPI can be highly reproducible, repeatable and robust across a broad spectrum of patient populations. Phase analysis can be based on the partial volume effect. The partial volume effect directs that left ventricle regional maximal counts in SPECT MPI images are nearly proportional to the myocardial wall thickness of the same region. Linear proportionality indicates that variation of regional maximal counts over a cardiac cycle represents myocardial wall thickening of the same region. The variation can be approximated using phase by employing harmonic functions to measure the onset of mechanical contraction.

Particularly, in phase analysis, gated SPECT MPI data can be reconstructed to produce a gated short-axis image. The SPECT MPI data can be taken at various temporal resolutions (e.g., 8 frames per cardiac cycle, 16 frames per cardiac cycle, 64 frames per cardiac cycle), and subsequent analysis and processing can enhance lower frame rates to identify or produce aspects typically associated with higher frame rates. Three dimensional sampling can be performed by searching each temporal frame to identify regional maximum counts. A first harmonic Fourier function can be used to approximate wall thickening data that in turn can be utilized to calculate a phase angle for each region. A phase distribution can be generated after phase angles are calculated for all regions (e.g., of the left ventricle). The phase distribution can be analyzed to determine uniformity or heterogeneity, which can be used to describe left mechanical synchrony or dyssynchrony. Phase standard deviation (PSD) and phase histogram bandwidth (PHB) are indices that can be generated and used to quantify global mechanical dyssynchrony.

While some imaging technologies may yield different or richer information than others in particular circumstances, alternatives or combinations should not be excluded as beyond the scope of the disclosure herein. Algorithms employed by, for example, analysis component 104 can utilize or be applied to alternative, limited or deprecated data to yield at least a portion of possible results, conclusions or suggestions in support of medical treatment as described herein.

In some embodiments, non-visual data, such as matrices, strings, or other encoded representation can be employed or used to store captured data or images relating to the patient. In some embodiments, visual data can be converted to nonvisual data for purposes of storage, analysis or others. In some embodiments, only a portion of data can be utilized (e.g., denser scar material as opposed to normal muscle tissue), stored or analyzed. While embodiments utilizing single-photon emission computed tomography (and/or other tomography techniques) can generate three-dimensional information, some embodiments can employ two-dimensional representations, alone or in combination with three-dimensional representations or other information relevant to identification, analysis, diagnosis and treatment of medical conditions cognizable utilizing the techniques described herein. In some embodiments, one or more proprietary or hybrid formats can be employed in conjunction with aspects herein.

Upon collection of information via imaging component 102, analysis component 104 can utilize the information. Utilization by analysis component 104 can facilitate determination as to a level of receptiveness for cardiac resynchronization therapy or other treatments related to electrical properties of a heart. Analysis component 104 can further identify distinguishing details related to a patient's heart and develop advanced, customized treatment related to implanting a cardiac resynchronization therapy device or other medical procedure.

In some embodiments, analysis component 104 can employ various algorithms to facilitate automated analysis of information captured or gathered via imaging component 102 (and/or other sources). Logic embodied in software or circuitry can identify features within tissue, as well as determining the effects of such features and the result of combined features and their interactions. For example, algorithms can identify scar tissue within a heart, including its size (dimensions including absolute and relative thickness in relation to the heart component in which it has developed), shape, orientation, density, conduction, and other aspects. Conditions such as asynchrony, and the particular type or measure of such conditions, can be identified and information relating to the conditions can be comprehensively developed. These algorithms can further consider a plurality of scar tissue deposits, alone and in combination, to determine the aggregate effects of the scar tissue in a heart. In some embodiments, the algorithms can additionally analyze healthy tissue such as non-scarred portions of the heart and surrounding tissue.

In some embodiments, an acceptable level of resolution can facilitate analysis despite imperfect or incomplete information. For example, while SPECT imaging cannot detect all scarring, undetectable scarring is unlikely to impact prognostication for CRT.

Analysis component 104 can be employed, for example, to determine or develop a condition of a patient's heart, whether the patient is a good candidate for cardiac resynchronization therapy, an optimized approach to applying cardiac resynchronization therapy, a guide for a surgeon to perform a procedure related to cardiac resynchronization therapy, and other aspects. Observed and/or computed factors apposite to such determinations and/or developments can include, but are not limited to, the impact of scar tissue in the heart on the heart's function, electrical conduction and other electrical properties, cardiac asynchrony, properties that continuously or periodically impact the heart's electrical function and interaction, or properties that continuously or periodically impact cardiac synchrony. Electrical conduction can also be considered independent from scarring or on non-scarred portions of the heart. Other properties that can be observed, computed, and/or applied in secondary, tertiary or subsequent algorithms can include blood flow (e.g., velocity, direction, volume), the thickness and movement of heart structures (e.g., chambers, valves, scar tissue and muscle tissue), the size and characteristics of heart chambers (e.g., overall size, wall thickness, cavity size, properties reflecting hypertension or high blood pressure), pumping function (e.g., ejection fraction), valve function (e.g., damage from infection or rheumatic fever, thickening, calcification, tearing, leakage, regurgitation, stenosis, narrowing, prolapse), related or unrelated conditions (e.g., areas of depressed movement, akinesia, dyskinesia, congenital defect, cardiomyopathy, aneurism, distension, excess pericardium fluid, clots, tumors, infection, blood pressure/volume), and other heart-related information.

In some embodiments, further patient characteristics can be employed when determining whether a patient is a good candidate for cardiac resynchronization therapy or resolving subsequent related determinations. For example, a patient's demographics (e.g., age, weight, gender, ethnicity) and health history (and/or medical record) (including, e.g., previous or current heart conditions, blood pressure, other illness or risk factors) can be provisioned by or provided to analysis component 104 for utilization during analysis. Specific, quantitative information on other organs or tissue can be included alternatively or in combination with qualitative health history or status information. Further patient characteristic information can be weighted, included or transformed for use in binary or gradient determinations resolved by algorithms employed in conjunction with the innovation(s) herein.

In some embodiments, analysis component 104 can display information captured utilizing imaging component 102 (and/or other sources) for manual analysis by a viewer. Displayed information can include three-dimensional models or pictures, two-dimensional pictures or representations, and/or non-pictorial data such as text, spreadsheets, and other representations facilitating analysis. Such manual analysis can be utilized exclusively or combined with automated analysis utilizing various algorithms or programs implemented by computers or circuitry.

In some embodiments, analysis component 104 can compare data from multiple sources to corroborate conclusions or develop a level of certainty in a determination. For example, single-photon emission computed tomography images can indicate the presence of scar tissue in a heart, including multiple deposits in several locations. If magnetic resonance imaging confirms these deposits, or an echocardiogram confirms substantial scarring, a level of certainty in the computed tomography images can be raised to indicate a multi-factor confirmation. Alternatively, if magnetic resonance imaging identifies a single, far smaller scar buildup, or an echocardiogram cannot identify any scarring, a level of certainty in the computed tomography images can be reduced. Analysis component 104 can additionally make additional, new or supplemental determinations by analyzing data from multiple sources, alone and in combination, to determine interactions between various sources and to increase confidence in multivariable functions or inferences.

In some embodiments, analysis component 104 can perform, among other analyses, a dyssynchrony analysis. Analysis component 104 can, in specific examples, execute dyssynchrony analysis of reconstructed, gated, short-axis SPECT images using medical image analysis software or circuitry configured for the same. In embodiments, dyssynchrony analysis can include automated left ventricle segmentation of gated SPECT images. Left ventricle segmentation algorithms can be trained across patient populations, permitting quantification of left ventricle volumes and ejection fraction to be improved in, for example, correlation and bias reduction. In embodiments, a patient myocardium can be divided into a plurality of segments per short-axis slice (e.g., 8 segments). Thereafter, a normalized time-activity curve can be derived for each segment. The time-activity curve can represent a radial thickening profile because the count density of a myocardial region is linearly related to myocardial thickness as a result of the partial volume effect during the cardiac cycle. One or more time-activity curves can be composed of a plurality of frames (e.g., 16 frames). Analysis component 104 can execute fast Fourier transform on the time-activity curve for each of a plurality of segments (e.g., 120 segments). A temporal peak of mechanical contraction during the cardiac cycle of each region can be determined using the phase of the first Fourier harmonic.

Using the calculations described above or others, analysis component 104 can quantify synchrony by the standard deviation of phase of peak mechanical activation, or phase standard deviation. In embodiments, higher values of PSD can denote less normal left ventricle contraction synchrony.

In some embodiments, analysis component 104 can further correct gating errors in gated SPECT imaging. An example of a gating error can include artifacts appearing as “flickering” or “flashing” in a reconstructed short-axis cine loop. Errors such as those in the example can be caused by count drop-off in the latter frames of the cardiac cycle due to inclusion of beats with a shorter “RR interval” (time between R waves) (or other arbitrary gate interval) than at the baseline that still fall within an acceptable window. These errors can be attributed to abnormal cardiac rhythm caused by conditions such as atrial fibrillation or frequent ectopic beats. In some embodiments of system 100, gating errors can have a relevant impact on SPECT analysis of left ventricle synchrony. For example, gating errors can cause a decrease in PSD, giving an incorrectly low indication of dyssynchrony. The mechanism by which this occurs is based on count drop-off in frames late in the cardiac cycle, which can alter the fit of the first sinusoidal harmonic to a perceived trough at the end of the cardiac cycle. The magnitude can be proportional to that of the drop-off. This effect can be magnified at longer PSDs, accentuating its impact on patients with diminished ejection fraction who are typically referred for the synchrony assay.

To correct for gating errors, embodiments of analysis component 104 can employ one or more multivariable models that resolve decay in PSD as a function of gating error magnitude. Corrective approaches employed by analysis component 104 can include normalizing the counts in affected frames, fitting a sinusoid to frames with preserved counts, and calculating the true PSD from the measured PSD and gating error severity. Where normalization is attempted, streak artifacts due to low signal-to-noise ratio in severely affected frames can be avoided by discarding such frames completely. Discarded frames can be replaced with, for example, duplicates of another frame (e.g., the first frame). Alternative means of reducing errors by normalization, curve-fitting, defining functions, deletion and duplication will be apparent to those skilled in the art on study of the disclosures herein.

Analysis component 104 can further perform a scar burden assessment. Scar burden can be a significant prognosticator of CRT success. In some embodiments, a scar burden cutoff (e.g., 15%, 40%) can be employed to determine a threshold at which to conduct CRT implantation. In other embodiments, scar burden can be calculated and factored into additional analysis for viability determinations.

In embodiments, SPECT methods can result in an overestimation of scar size in the left ventricle (LV). As a result of the partial volume effect, non-transmural scar appears to diminish the tracer uptake across the entire LV wall. The severity of uptake diminishment across the LV wall is linearly related to scar transmurality. The use of strict cutoffs and calculation of scar extent, whether referenced to the “hottest” LV region or based on a normal database, can overestimate scar burden due to inclusion of the entire wall in cases where only endocardial scar exists. However, by using both scar extent and severity of tracer uptake diminishment to calculate scar burden, analysis component 104 can avoid or mitigate such overestimation. Additionally, the use of algorithms automating myocardium-at-risk segmentation (e.g., Segment MaR algorithm utilizing knowledge on perfusion territories) by analysis component 104 can improve the delineation of scar extent without the use of a normal database by normalizing the counts in the thinner basal and apical regions and using an a priori model of coronary artery distribution.

Analysis component 104 can utilize dyssynchrony and scar burden results to develop viability information. In an embodiment, viability information can be incorporated with left ventricle dyssynchrony assessment by filtering out regions with scar encompassing more than a threshold amount of the area (e.g., 50%). Exclusion of non-viable regions can improve CRT prognosis because pacing corrects activation sequences only in viable myocardium. Scar tissue does not contract, and thus cannot be corrected by CRT. Further, in regions with extensive scarring, phase of contraction cannot be calculated (because these areas do not thicken), interfering with dyssynchrony assessment. In some patients, regions adjacent to scarring can activate early and late, in a manner suggesting that phase result in these regions is derived primary from noise. Thus, exclusion can improve analysis related to CRT viability, and better resolve particular CRT parameters and implantation specifics in viable patients.

In some embodiments, a strong correspondence between SPECT dyssynchrony and scar burden can be observed. This relationship is minimized or disappears after “filtering” of scarred regions, suggesting that the strong correspondence prior to filtering can be attributable to signal from the scar itself. Analysis component 104 can employ a filtering cutoff (e.g., greater than 50% transmurality) that can correspond to irreversibly damaged, non-thickening myocardium precluding response to CRT when present at the tip of the LV lead. Prior to filtering, patients with ischemic cardiomyopathy (ICM) can have more dyssynchrony than patients with non-ICM (NICM). Given that patients with ICM are less likely than those with NICM to respond to CRT, the effect of scar on SPECT dyssynchrony can result in suboptimal performance of PSD alone for the prediction of CRT outcomes. Scar and dyssynchrony can be utilized by analysis component 104 for outcome prediction by using separate cutoffs for each. However, integration of viability and dyssynchrony information as proposed in some embodiments herein provides a direct metric of activation timing in the non-scarred myocardium.

In embodiments, further processes performed by analysis component 104 can include implementation of a segmental delay vector (SDV) that assesses an activation pattern in non-scarred myocardium. The vector can be constructed in some embodiments by representing each segmental phase as a three-dimensional vector oriented toward the center of the left ventricle and summing each segmental vector to obtain a net magnitude and direction of regional activation delay. In such embodiments, regional clustering of delayed contraction can be associated with a large delay vector, with the vector oriented toward the area of latest activation. In embodiments, regions of scar and/or low amplitude in which phase cannot be reliably assessed can be replaced with a vector with a magnitude equal to the average phase over the entire left ventricle. Other embodiments, such as alternatives for calculation vectors and/or replacement of regions in which phase cannot be reliably assessed, will be apparent to those skilled in the art in view of the innovation, and such example solutions should not be taken to limit the breadth of the disclosure but rather provide limited possible embodiments capturing the spirit and thrust of such. In embodiments employing one or more segmental delay vectors, the magnitude of the SDV oriented towards the lateral wall as assessed by SDV was significantly larger in CRT responders, and can be used, at least in part, to predict viability response to CRT.

Analysis component 104 can additionally be utilized to develop a prognosis based on a “dyssynchrony reserve” using dobutamine. The presence of contractile reserve, or the increase in ejection fraction in response to low-dose dobutamine infusion, can be a predictive factor for CRT response. Patients without contractile reserve tend to have severe LV dysfunction, and these patients can fail to benefit from CRT because their hearts are too sick to recover with resynchronization alone. In patients with poor LV thickening, activation pattern is technically difficult to discern. SPECT imaging during dobutamine infusion, in patients whose hearts harbor contractile reserve, makes activation pattern significantly more apparent. In some embodiments, analysis component 104 can identify the presence of “dyssynchrony reserve,” or a significant SDV during dobutamine infusion. This can be prognostic because it demonstrates the presence of both contractile reserve and an activation delay in the lateral wall.

While several techniques set forth above describe functions that can be performed by analysis component 104, it is appreciated that various embodiments can include one or more such functions in various combinations, and every embodiment of system 100 need not include all aspects described above, in their narrowest form or at all. Further, such techniques can be employed elsewhere throughout aspects of the innovation, where appropriate and as will be appreciated by skilled artisans upon study of the disclosure.

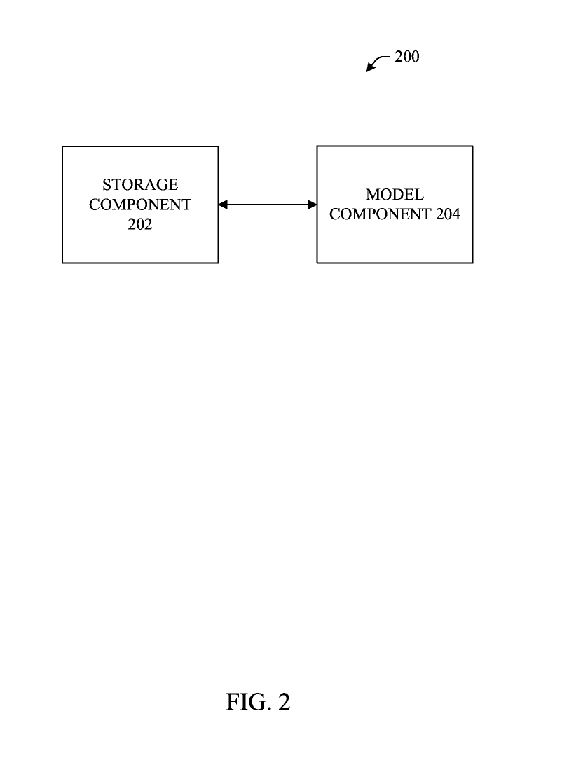

Turning now to FIG. 2, illustrated is an example system 200 including storage component 202 and model component 204. Storage component 202 can include, for example, information related to a patient's healthcare. Model component 204 can utilize information, accessed at least in part via storage component 202, to generate a model used in conjunction with medical procedures.

Storage component 202 can store, in some embodiments, data related to a heart and heart conditions relevant to the efficacy and optimal means of administering cardiac resynchronization therapy. Data can be collected, for example, via imaging components and/or techniques like those described with respect to FIG. 1 and elsewhere herein.

Model component 204 can provide a display or other information related at least in part to the information in storage component 202. In some embodiments, model component 204 can display a preexisting model or generate a new model based on information from storage component 202. The model can be, for example, a model of a human heart (and/or tissue around or related to the heart) used to provide a medical practitioner with additional patient information. In some embodiments, the model can be 3-dimensional, and permit manipulation for multiple views or cross-sections. In some embodiments, the model can be used in conjunction with a “virtual roadmap” for treatment, providing precise details on an optimized method for conducting a treatment. In embodiments, the model can be the treatment roadmap itself.

In some embodiments, model component 204 can present a heart model for integration in an operating room. The heart model can provide precise details relating to the heart and other patient aspects to assist a surgeon or other medical practitioner with the implantation of a cardiac resynchronization therapy device.

In some embodiments, storage component 202 can be connected, directly or remotely, to various imaging and analysis components, and act as an intermediary between imaging and analysis techniques and model component 204. In alternative embodiments, storage component 202 can be included within another component. In some embodiments, storage component 202 can provide temporary storage facilitating data from other components or sources to pass directly through to model component 204 (e.g., in embodiments where imaging systems can be included).

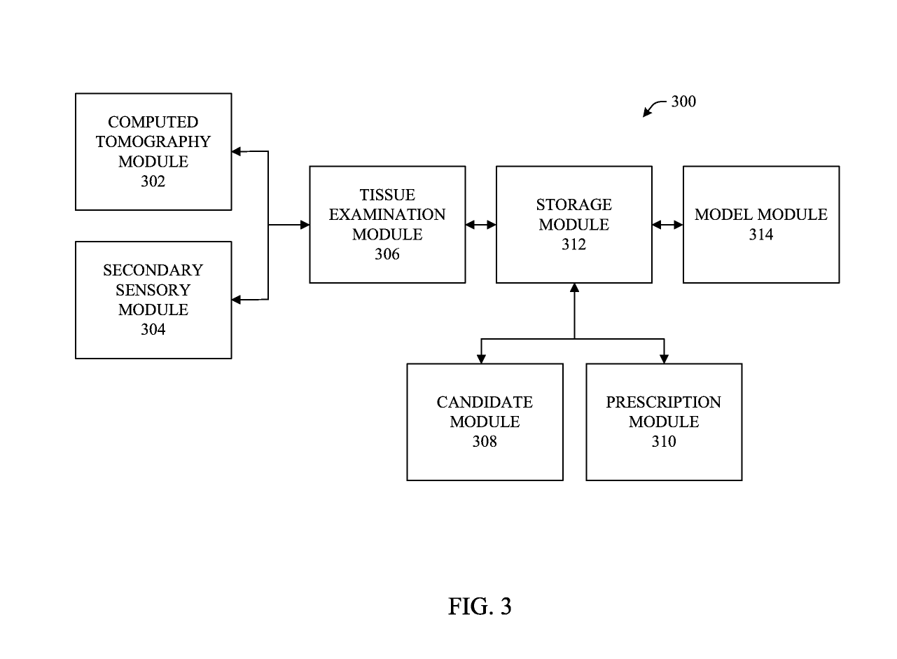

Turning now to FIG. 3, illustrated is an example system 300 including a series of modules relating to a heart model and associated treatment. System 300 can include computed tomography module 302 and secondary sensory module 304. In some embodiments, the heart model can be “multi-imaged,” meaning the heart is diagnosed, measured or sensed via two or more imaging or diagnostic techniques. Computed tomography module 302 and secondary sensory module 304 can provide information to tissue examination module 306. Tissue examination module 306 interacts with storage module 312. Candidate module 308 and prescription module 310 also interact with storage module 312. Model module 314 can access storage module 312, to retrieve information to generate a model, to store a generated model, and for other purposes.

Computed tomography module 302 can gather tomographic information, including, but not limited to, three-dimensional image data related to a patient's heart via single-photon emission computed tomography. In embodiments, alternative or supplemental tomography technologies can be employed. Secondary sensory module 304 can gather additional data related to the patient's heart for imaging, analysis, diagnosis, treatment, modeling, and other uses. In some embodiments, secondary sensory module can be an echocardiogram or data therefrom. In other embodiments, secondary sensory module can be information from or equipment performing magnetic imaging, electrocardiography, infrared imaging, x-ray or other nuclear medicine imaging, various sonography techniques, and others. In some embodiments, secondary sensory module can be a plurality of modules, including combinations of technologies described above and others. In alternative embodiments, secondary sensory module 304 can be optional or excluded.

Tissue examination module 306 can perform analysis on the information from computed tomography module 302 and secondary sensory module 304. Tissue examination module 306 can determine, for example, the presence and character of scar tissue within a patient's heart. After identifying scar tissue, tissue examination module 306 can calculate the impact of the scar tissue on the heart function and overall characteristics or conditions (e.g., asynchrony, electrical conduction). Tissue examination module 306 can provide its results to storage module 312, as well as provide original, unprocessed information from computed tomography module 302 and secondary sensory module 304 for storage utilizing storage module 312. In some embodiments, storage module 312 can interact directly with computed tomography module 302, secondary sensor module 304, and other modules in system 300, and stores such information upon collection.

Candidate module 308 and prescription module 310 can utilize and process results from tissue examination module 306 or unprocessed information from computed tomography module 302, secondary sensory module 304, and/or other sources. Candidate module 308 can employ one or more procedures to determine, for example, a rating regarding a patient's likelihood of responding to a heart treatment. In some embodiments, the heart treatment can be cardiac resynchronization therapy. In some embodiments, a plurality of heart treatments can be assessed simultaneously.

In particular embodiments, candidate module 308 can evaluate left ventricle mechanical dyssynchrony parameters. Candidate module 308 can receive (e.g., from tissue examination module 306, storage module 312, or other sources) or calculate PSD and/or PHB. Optimal cutoff values for PSD and PHB can be provided or determined, and the calculated PSD and/or PHB can be compared to the optimal cutoff values. In some embodiments, an optimal cutoff value for PSD can be between 35° and 50°, and an optimal cutoff value for PHB can be between 125° and 145°. In some embodiments, where a patient PSD or PHB is below a cutoff value, candidate module returns a negative response regarding whether the patient is a good candidate for CRT. In other embodiments, an optimal cutoff value can establish a maximum rather than a minimum. In embodiments, PSD and/or PHB can be evaluated as cutoffs in isolation, or be evaluated together. Where PSD or PHB are evaluated in isolation, either value can render a patient a good candidate or bad candidate (e.g., either one meeting threshold results in good candidate, or either one failing threshold results in bad candidate). Alternatively, both must be pass or fail the threshold to return a particular response. Various other means of determining candidacy based on these and other techniques described herein will be apparent to those skilled in the art upon review of the disclosure.

Further phase analysis can be employed that quantifies regional mechanical activation. A multi-segment (e.g., 7 segments) model can divide a phase polar map generated from a phase distribution into apex, anterior, lateral, inferolateral, inferior, septal, and anteroseptal regions. The six regions other than the apex divide the mid-basal left ventricle evenly, and the mean phases of the six regions can be compared. The site of latest mechanical activation can be identified as the region with the largest mean. Such phase analysis techniques can be highly reproducible in identifying site of latest mechanical activation.

Prescription module 310 can employ one or more procedures to determine, for example, a specific prescription, medical procedure, or customized fashion for administering treatment, based at least in part on results from tissue examination module 306 or unprocessed information from computed tomography module 302, secondary sensory module 304, and/or other sources.

As phase analysis can be utilized to assess LV mechanical dyssynchrony and site of latest mechanical activation, and SPECT MPI can be utilized for assessment of myocardial scar, this imaging process can facilitate comprehensive evaluation of parameters for optimizing CRT. Prescription module 310 can utilize a comprehensive evaluation to resolve particular aspects of treatment in a patient. For example, LV lead placement can be determined by prescription module 310. This information can be provided, alone or in combination with other prescription information, to a practitioner or for storage by prescription module 310. In patients currently selected for CRT treatment, baseline LV mechanical dyssynchrony can be evaluated and performed with the LV pacing lead placed in the site of latest mechanical activation with viable myocardium. By employing an optimal LV lead position, very high CRT response rates can be achieved. Comprehensive models of this nature can have a high positive predictive value in predicting CRT response and can significantly improve CRT response rate if it is used to screen the patients currently indicated for CRT and to guide LV lead placement. This is just one example of aspects that can be facilitated by prescription module 310, alone or in combination with candidate module 308, and is intended to suggest aspects of embodiments, rather than provide an exhaustive catalog of all prescriptive function.

In some embodiments, candidate module 308 and prescription module 310 are related or share a chronological connection. In such embodiments, prescription module 310 can condition its action or modify its prescription based at least in part on a result from candidate module 308. In alternative embodiments, candidate module 308 and prescription module 310 can act independently, acting simultaneously or without reference to the other. In embodiments where candidate module 308 and prescription module 310 act in an unrelated manner, prescription module 310 can provide a prescription (or provide no prescription) without basing a prescription on a positive or negative recommendation (or lack thereof) from candidate module 308.

Candidate module 308 and prescription module 310 can provide determinations for aggregation with other information to storage module 312. Storage module 312 can be local, remote, distributed, and/or combinations thereof. In some embodiment, one or more modules within system 300 can contain or be allocated its own storage.

In some embodiments, one or more of tissue examination module 306, candidate module 308, and/or prescription module 310 can be effected using algorithms, procedures or equipment associated with computer or circuit logic from the proprietary SmartPace™ system.

Model module 314 can access storage module 312 to generate a patient model based at least in part on information from computed tomography module 302 and/or secondary sensory module 304. In at least one embodiment, model module 314 can access computed tomography module 302 and/or secondary sensor module 304 directly or indirectly. In some embodiments, model module can access tissue examination module 306, candidate module 308 and/or prescription module 310 directly or indirectly. Model module 314 can thus create a more accurate model of a patient or a portion of a patient, including the results of analyses, details or relationships within the patient or discerned by other modules of system 300. In some embodiments, model module 314 can run concurrently with computed tomography module 302 and/or secondary sensory module 304, providing a real-time model reflecting the current state of the patient.

Model module 314 can be used to display a patient treatment or prescription for treatment. For example, a patient can be determined to be a good candidate for an artificial pacemaker implant, and a specific prescription can be determined for the pacemaker implant. The prescription can include information on a preferred pacemaker, optimal pacemaker settings, and the particular way of implanting the pacemaker in terms of location in relation to scar tissue or other heart abnormalities in order to achieve an optimized result. Model module 314 can display a two- or three-dimensional model displaying the particular patient's heart, including its idiosyncrasies and abnormalities. In some embodiments, model module 314 can further display the prescription, providing a “virtual roadmap” for a surgeon or other practitioner to perform the therapy in accordance with the specific patient details. In this way, treatments can be customized. Rather than just performing a heart treatment on a heart that shares similarities with the clinically idealized heart, a medical practitioner can be provided specific information and detailed, unique solutions for the exact heart being treated.

Model module 314 can include or facilitate display of a model generated by model module 314 based on information gathered and developed via system 300. In some embodiments, model module 314 includes a physical display. In alternative embodiments, model module 314 includes software, routines, or procedures to display the model on a device (e.g., computer). In other embodiments, model module 314 generates a model in a proprietary format for use with one or more devices including or capable of coupling to displays. In still other embodiments, model module 314 generates a model in a known or generic format that can be displayed on another device, but does not participate in the display beyond providing data to be displayed.

In some embodiments, system 300 can include various communication means to facilitate data exchange between modules or with external systems. One or more wired or wireless network connections can be included in or accessible to system 300. Various other wired and wireless electronic communication means, including not only digital but also audio and visual information, can also establish physical or logical connections between the modules of system 300 and/or external systems or modules.

System 300 can also be utilized to optimize implantable cardioverter defibrillator (ICD) therapy devices. Phase analysis SPECT MPI techniques can be used to discern LV mechanical dyssynchrony severity that be used, at least in part, to discern predictive and prescriptive information relating to ICD implantation and function.

In some embodiments, at least one of tissue examination module 306, candidate module 308, and prescription module 310 can utilize diastolic dyssynchrony assessments in one or more analyses or determinations. While aspects herein have primarily focused in onset of mechanical contraction, an alternative or complementary identification and study of onset of mechanical relaxation can be used to measure diastolic dyssynchrony. Such results can be used, alone or in combination with others, to more thoroughly analyze heart function, procedure candidacy and individualized procedure prescriptions.

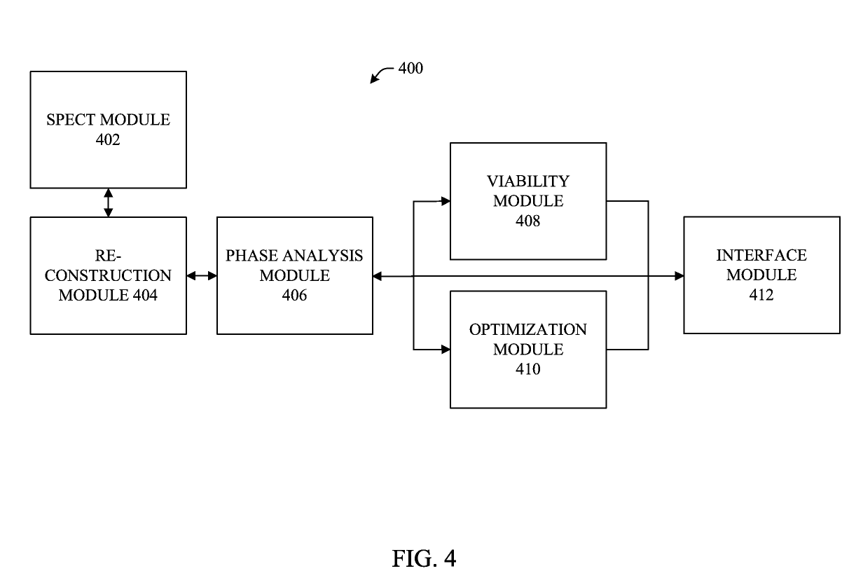

Turning now to FIG. 4, illustrated is an example system 400 illustrating an embodiment for providing viability and optimization information related to a heart. System 400 can include SPECT module 402, reconstruction module 404, phase analysis module 406, viability module 408, optimization module 410, and interface module 412.

SPECT module 402 can collect data related to a patient heart via gated SPECT MPI short-axis imaging in accordance with aspects herein. Data captured using SPECT module 402 can be provided to reconstruction module 404, which reconstructs and reorients the data to create a gated short-axis image of the patient heart. Phase analysis module 404 can analyze the gated short-axis image to produce three-dimensional representations of the patient heart. The representations produced by phase analysis module 404 can include, but are not limited to, phase distributions as polar maps and/or histograms.

Upon production of the phase distribution, and in some embodiments after further analysis by phase analysis module 404, both original and developed data can be provided to viability module 408, optimization module 410, and interface module 412. Viability module 408 and optimization module 410 can perform analyses to determine the viability of a therapy to the patient heart (e.g., likelihood of patient responsiveness to CRT) and means for optimizing the therapy to the patient heart (e.g., configuration of CRT device and lead placement) as described throughout this disclosure. In some embodiments, global or regional phase data related to the patient heart can be compared to thresholds to determine likelihoods of success and details relating to therapy execution.

Interface module 412 can receive information from phase analysis module 406, as well as results from viability module 408 and optimization module 410 to present to a practitioner or other entity. Thus, information related to a patient's heart, three-dimensional models, and calculated viability and optimization information can be provided in a flexible, usable manner. In some embodiments, interface module 412 can integrate the information discovered and developed via system 400 to provide a “virtual roadmap” for patient treatment that guides a practitioner through the decision to prescribe CRT, as well as preparation, implantation and use of the therapy and/or device thereafter.

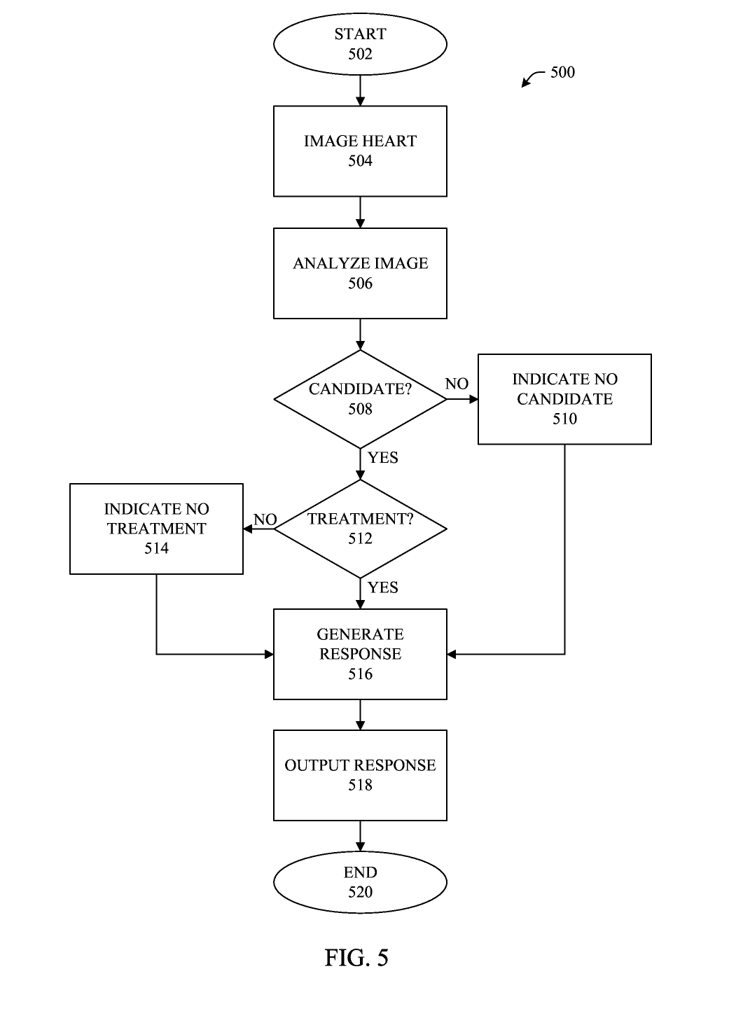

Turning now to FIG. 5, illustrated is an example methodology 500 for determining patient heart treatment. The methodology begins at 502 and proceeds to 504 where a heart can be imaged. Imaging can occur, for example, by single photon emission computed tomography. In various embodiments, imaging the heart at 504 can include other techniques described herein, and/or multiple and combinations of techniques.

Once imaging of the heart is complete, the image can be analyzed at 506. Image analysis at 506 can include, but is not limited to, location of scar tissue or other aberrations or abnormalities in or around the heart. Image analysis at 506 can also include identifying characteristics and exact details of the heart, such as the precise dimensions, orientations and relationships of different parts of the heart. In some embodiments, image analysis at 506 can also invoke extrinsic information relevant to determinations but gathered from sources other than imaging of the heart at 504. For example, at 506, patient demographics, health history, and other information can be analyzed for use in determinations or for quick access by a practitioner.

At 508, a determination is made as to whether the patient associated with the heart image taken at 504 is a good candidate for a treatment or procedure. Various computer- or circuit-implemented algorithms can be employed, based at least in part upon the analysis of the heart image, to resolve this determination. Such a determination can be rendered in a “yes-or-no” format, or be presented on a scale or scored. If the answer is yes, or a score exceeds a threshold, methodology 500 proceeds to 512. When proceeding to 512, the determination can be stored for use in generating a response at 516. If the patient is determined not to be a good candidate at 508, meaning the determination is “no” or falls below a threshold, methodology 500 proceeds to 510, where an indication is stored that the patient is not a good candidate for use in generating a response at 516.

In the illustrated embodiment, after indicating that the patient is not a good candidate for treatment at 510, methodology 500 proceeds to 516. However, in some embodiments, methodology 500 can still analyze and resolve if a particular treatment should be modified or performed at 512 even if the determination at 508 is returned in the negative. In alternative embodiments, methodology 500 can proceed through multiple simultaneous paths, and the determinations at 508 and 512 can be performed independently with no dependence on the other. Other techniques for organizing the steps of methodology 500 without deviating from the scope and spirit of the innovation will be apparent to those skilled in the art in view of the disclosures herein.

At 512, a determination is made as to whether a specific treatment should be pursued. The determination at 512 can include discovering or solving for the specific treatment to be pursued. In an embodiment, the determination at 512 can recommend a customized medical procedure, performed in accordance with the specific characteristics of the patient's heart imaged at 504. In one example, a particular configuration for a cardiac resynchronization therapy device and specific details of a non-generic implantation procedure can be determined at 512 and included in results at 516. In some embodiments, a determination can be made that no treatment, or no custom treatment, should be pursued, and methodology 500 can proceed to indicate that no specialized treatment is resolved at 514.

After the determination at 512, either directly or via 514, methodology 500 proceeds to generate a response at 516. The response at 516 can include one or more solutions related to one or more medical procedures. Solutions can include suggestions or scores regarding the patient's candidacy for a particular procedure, solutions or customizations and/or modifications for medical procedures, and other information. Upon generation and/or aggregation of the response at 516, the response can be output at 518. Output at 518 can include displaying the presenting (e.g., visually, audibly) the output to a medical practitioner or other entity. Upon output of the solutions based on the heart image sensed and constructed at 504, methodology proceeds to its end at 520.

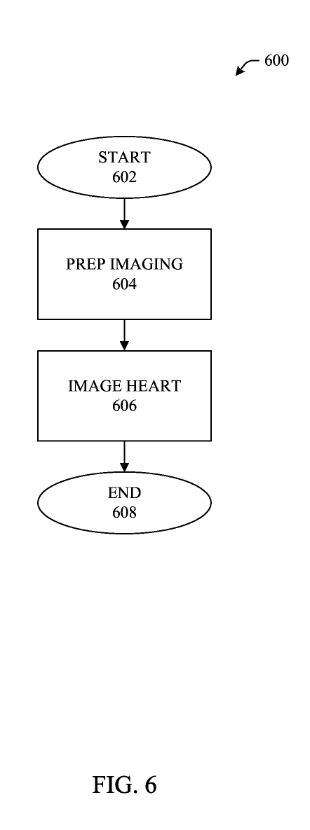

Turning now to FIG. 6, illustrated is an example methodology 600 for imaging a patient heart. At 602, methodology 600 begins, and proceeds to prepare for imaging of the patient heart at 604. Preparations can included, but are not limited to, performing preparation work on the patient (e.g., apply contrast dye if magnetic resonance imaging with contrast dye is being used, for example, with secondary sensory module 304), preparing equipment (e.g., turning on and preparing a single photon emission computer tomography system), and associating resources (e.g., powering or accessing storage to retain the results of imaging). After preparations are complete, the heart can be imaged at 606. Imaging the heart can include, for example, one or more single photon emission computed tomographic procedures. Imaging captured at 606 can be saved, displayed, or forwarded to other components or systems.

In some embodiments, imaging at 606 includes analysis, such as the identification of different tissue types, location and significance of abnormalities, diagnosis of conditions, analysis of properties (e.g., electrical conduction through one or more portions or the entire organ, asynchrony), determinations about a patient's receptiveness to a treatment (e.g., cardiac resynchronization therapy), customizations to a treatment (e.g., how to best configure and implant a cardiac resynchronization therapy device), and so forth. In some embodiments, analysis can be completed using algorithms and modules associated with the proprietary SmartPace™ platform. Upon completion of the imaging at 606, methodology 600 can end.

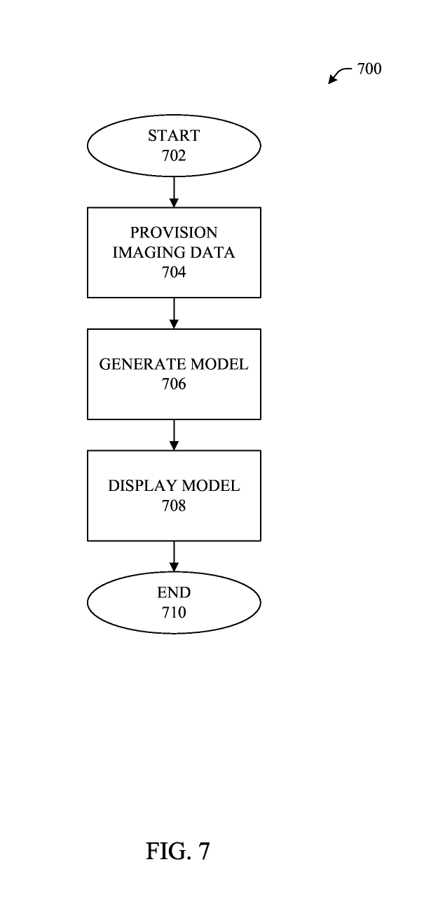

Turning now to FIG. 7, illustrated is an example methodology 700 for generating a model of a patient heart. At 702, methodology 700 begins and proceeds to provision imaging data at 704. Data provisioned at 704 can additionally include information resultant to analyses on imaging data, as well as patient information such as demographics and health history. In some embodiments, supplemental information, or information from multiple sources, can be gathered at 704. Sources of information can include single photon emission computed tomography imaging. Additional sources can include, but are not limited to, echocardiograms, other sonography, magnetic resonance imaging, electrocardiograms, x-ray, and others.

At 706, a model can be generated based on data provisioned at 704. The model can be, for example, one or more of, or combinations of, two- and three-dimensional images including particular details of the imaging data. For example, a model can include a patient's heart including the particular geometry, distinctions and abnormalities, such as scarring or other ongoing conditions. In some embodiments, a model generated at 706 can also predict both characteristics that cannot be directly tested (e.g., electrical conduction in one or more portions of the heart, asynchrony), reactions in areas based on treatment or stimulus, and other predictive information not directly measured or apparent from one or more images, diagnostic efforts, and/or known medical information details.

In some embodiments, step(s) at 706 can include analysis that tailors a specific medical device or procedure (e.g., a cardiac resynchronization therapy device, a surgery to implant a cardiac resynchronization device) to the patient's exact specifics. For example, at 706, a determination can be made to employ a particular device or device setting (e.g., device sensitivity, lead impedance, voltage, pulse width, and others) to treat a patient. Another example, that can be used alone or in combination with the former example, can include particular features of the implantation surgery (e.g., a specific site to attach leads, avoidance of scar tissue, and others).

The model generated at 706 can additionally provide a “virtual roadmap” for a medical practitioner to employ the device or perform a procedure like those described herein. Device or procedure details can be determined during model generation at 706, or retrieved at 704 with other stored data. In some embodiments, combinations of both can occur as predetermined treatment details are further developed upon model generation.

Once model generation is complete at 706, the model can be displayed at 708. Display of the model can be accomplished by direct display to a local device, display to a remote device, display through a proprietary device or module configured to manage the model information, or display through a known file extension or information format via a preexisting protocol and common software or hardware modules. After displaying the model, methodology 700 can end at 710.

While the foregoing figures are directed toward diagnosis and treatment of heart conditions, particularly with respect to cardiac resynchronization therapy, it is appreciated that the techniques described herein can be applied to other medical conditions and portions of bodies for treatment. For example, tomographic techniques can be employed on other parts of the body, including other organs or joints, and algorithms focused in identifying and analyzing scar tissue within the heart can be applied or modified for application for identification and analysis of scar tissue or other conditions (e.g., ulcers, arterial obstruction, tumors). In this way, the techniques set forth herein can be employed to augment the way diagnoses and treatments are applied in a variety of medical fields, and can be utilized to develop more specific, focused therapies based on more specific details of an individuals' health than have been employed in prior practice.

Further, while the figures above are focused toward cardiac resynchronization therapy, the selection, configuration, application, and implantation of various artificial pacemaker technologies or heart implants can be accomplished and improved in accordance with disclosures herein. Those skilled in the art will appreciate how modifications, understandable in view of the disclosures herein, can be effected to permit optimization of a variety of artificial pacemaker treatments or other procedures on patient hearts. For example, the disclosure should not be interpreted, unless expressly provided to the contrary, to preclude the use of systems and methods described herein in conjunction with various cardiac resynchronization therapies, electrode or electrical stimulation pacing, percussive pacing, transcutaneous pacing, temporary or long-term epicardial pacing, and/or temporary or long-term transvenous pacing, and others. Biventricle or monoventricle configurations, or single-chamber, dual-chamber, and/or rate responsive pacemaker variants, and other types of pacemakers can all potentially be cognized and optimized under at least portions of the disclosures herein.

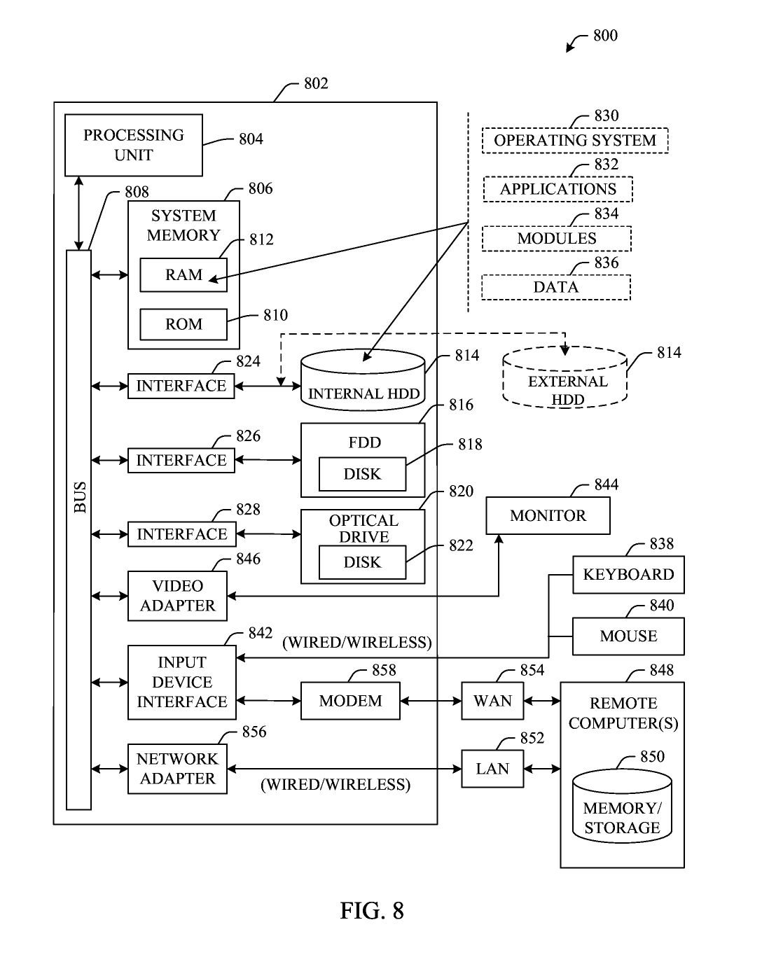

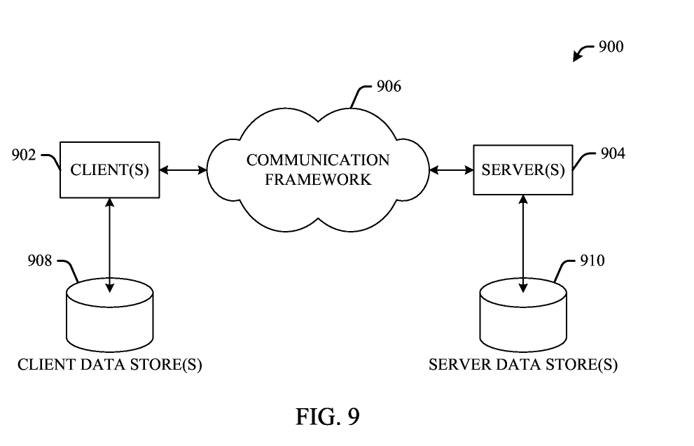

FIG. 8 illustrates a brief general description of a suitable computing environment wherein the various aspects of the subject innovation can be implemented, and FIG. 9 illustrates a schematic diagram of a client—server-computing environment wherein the various aspects of the subject innovation can be implemented.

With reference to FIG. 8, the exemplary environment 800 for implementing various aspects of the innovation includes a computer 802, the computer 802 including a processing unit 804, a system memory 806 and a system bus 808. The system bus 808 couples system components including, but not limited to, the system memory 806 to the processing unit 804. The processing unit 804 can be any of various commercially available processors. Dual microprocessors and other multi-processor architectures may also be employed as the processing unit 804.

The system bus 808 can be any of several types of bus structure that may further interconnect to a memory bus (with or without a memory controller), a peripheral bus, and a local bus using any of a variety of commercially available bus architectures. The system memory 806 includes read-only memory (ROM) 810 and random access memory (RAM) 812. A basic input/output system (BIOS) is stored in a non-volatile memory 810 such as ROM, EPROM, EEPROM, which BIOS contains the basic routines that help to transfer information between elements within the computer 802, such as during start-up. The RAM 812 can also include a high-speed RAM such as static RAM for caching data.

The computer 802 further includes an internal hard disk drive (HDD) 814 (e.g., EIDE, SATA). Alternatively or in addition, an external hard disk drive 815 may also be configured for external use in a suitable chassis (not shown), a magnetic disk drive, depicted as a floppy disk drive (FDD) 816, (e.g., to read from or write to a removable diskette 818) and an optical disk drive 820, (e.g., reading a CD-ROM disk 822 or, to read from or write to other high capacity optical media such as the DVD). The hard disk drives 814, 815 magnetic disk drive 816 and optical disk drive 820 can be connected to the system bus 808 by a hard disk drive interface 824, a magnetic disk drive interface 826 and an optical drive interface 828, respectively. The interface 824 for external drive implementations can include Universal Serial Bus (USB), IEEE 1394 interface technologies, and/or other external drive connection technologies.

The drives and their associated computer-readable media provide nonvolatile storage of data, data structures, computer-executable instructions, and so forth. For the computer 802, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the innovation.

A number of program modules can be stored in the drives and system memory 806, including an operating system 830, one or more application programs 832, other program modules 834 and program data 836. All or portions of the operating system, applications, modules, and/or data can also be cached in the RAM 812. It is appreciated that the innovation can be implemented with various commercially available operating systems or combinations of operating systems.

A user can enter commands and information into the computer 802 through one or more wired/wireless input devices, e.g., a keyboard 838 and a pointing device, such as a mouse 840. Other input devices (not shown) may include a microphone, an IR remote control, a joystick, a game pad, a stylus pen, touch screen, or the like. These and other input devices are often connected to the processing unit 804 through an input device interface 842 that is coupled to the system bus 808, but can be connected by other interfaces, such as a parallel port, an IEEE 1394 serial port, a game port, a USB port, an IR interface, et cetera

A monitor 844 or other type of display device is also connected to the system bus 808 via an interface, such as a video adapter 846. In addition to the monitor 844, a computer typically includes other peripheral output devices (not shown), such as speakers, printers, et cetera

The computer 802 may operate in a networked environment using logical connections via wired and/or wireless communications to one or more remote computers, depicted as remote computer(s) 848. The remote computer(s) 848 can be a workstation, a server computer, a router, a personal computer, portable computer, microprocessor-based entertainment appliance, a peer device or other common network node, and typically includes many or all of the elements described relative to the computer 802, although, for purposes of brevity, only a memory/storage device 850 is illustrated. The logical connections depicted include wired/wireless connectivity to a local area network (LAN) 852 and/or larger networks, e.g., a wide area network (WAN) 854. Such LAN and WAN networking environments are commonplace in offices and companies, and facilitate enterprise-wide computer networks, such as intranets, all of which may connect to a global communications network, e.g., the Internet.

When used in a LAN networking environment, the computer 802 is connected to the local network 852 through a wired and/or wireless communication network interface or adapter 856. The adapter 856 may facilitate wired or wireless communication to the LAN 852, which may also include a wireless access point disposed thereon for communicating with the wireless adapter 856.

When used in a WAN networking environment, the computer 802 can include a modem 858, or is connected to a communications server on the WAN 854, or has other means for establishing communications over the WAN 854, such as by way of the Internet. The modem 858, which can be internal or external and a wired or wireless device, is connected to the system bus 808 via the serial port interface 842 as depicted. It should be appreciated that the modem 858 can be connected via a USB connection, a PCMCIA connection, or another connection protocol. In a networked environment, program modules depicted relative to the computer 802, or portions thereof, can be stored in the remote memory/storage device 850. It will be appreciated that the network connections shown are exemplary and other means of establishing a communications link between the computers can be used.

The computer 802 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11(a, b, g, et cetera) to provide secure, reliable, fast wireless connectivity. A Wi-Fi network can be used to connect computers to each other, to the Internet, and to wired networks (which use IEEE 802.3 or Ethernet).

FIG. 9 is a schematic block diagram of a sample-computing environment 900 that can be employed for practicing aspects of the aforementioned methodology. The system 900 includes one or more client(s) 902. The client(s) 902 can be hardware and/or software (e.g., threads, processes, computing devices). The system 900 also includes one or more server(s) 904. The server(s) 904 can also be hardware and/or software (e.g., threads, processes, computing devices). The servers 904 can house threads to perform transformations by employing the components described herein, for example. One possible communication between a client 902 and a server 904 may be in the form of a data packet adapted to be transmitted between two or more computer processes. The system 900 includes a communication framework 906 that can be employed to facilitate communications between the client(s) 902 and the server(s) 904. The client(s) 902 are operatively connected to one or more client data store(s) 908 that can be employed to store information local to the client(s) 902. Similarly, the server(s) 904 are operatively connected to one or more server data store(s) 910 that can be employed to store information local to the servers 904.

What has been described above includes examples of the various versions. It is, of course, not possible to describe every conceivable combination of components or methodologies for purposes of describing the various versions, but one of ordinary skill in the art may recognize that many further combinations and permutations are possible. Accordingly, the subject specification intended to embrace all such alterations, modifications, and variations that fall within the spirit and scope of the appended claims.

It is appreciated that, while aspects of the subject innovation described herein focus in wholly-automated systems, this should not be read to exclude partially-automated or manual aspects from the scope of the subject innovation. Practicing portions or all of some embodiments manually does not violate the spirit of the subject innovation.

What has been described above includes examples of the various aspects. It is, of course, not possible to describe every conceivable combination of components or methodologies for purposes of describing the various aspects, but one of ordinary skill in the art may recognize that many further combinations and permutations are possible. Accordingly, the subject specification intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects. In this regard, it will also be recognized that the various aspects include a system as well as a computer-readable medium having computer-executable instructions for performing the acts and/or events of the various methods.

In addition, while a particular feature may have been disclosed with respect to only one of several implementations, such feature may be combined with one or more other features of the other implementations as may be desired and advantageous for any given or particular application. To the extent that the terms “includes,” and “including” and variants thereof are used in either the detailed description or the claims, these terms are intended to be inclusive in a manner similar to the term “comprising.” Furthermore, the term “or” as used in either the detailed description of the claims is meant to be a “non-exclusive or”.

Furthermore, as will be appreciated, various portions of the disclosed systems and methods may include or consist of artificial intelligence, machine learning, or knowledge or rule based components, sub-components, processes, means, methodologies, or mechanisms (e.g., support vector machines, neural networks, expert systems, Bayesian belief networks, fuzzy logic, data fusion engines, classifiers, and so forth). Such components, inter alia, can automate certain mechanisms or processes performed thereby to make portions of the systems and methods more adaptive as well as efficient and intelligent. By way of example and not limitation, the aggregation of password rules can infer or predict support or the degree of parallelism provided by a machine based on previous interactions with the same or like machines under similar conditions. As another example, touch scoring can adapt to hacker patterns to adjust scoring to thwart successful approaches.

In view of the exemplary systems described supra, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers. The term article of manufacture, as used herein, is intended to encompass a computer program accessible from any computer-readable device, carrier, or media.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

What is claimed is:

1. A system for selection of patients for a heart therapy, comprising:
an examination component that identifies one or more characteristics of a heart based at least in part on data derived from single proton emission computed tomography imaging; and
an analysis component that calculates a level of receptiveness to the heart therapy based at least in part on the at least one characteristic of the heart.

2. The system of claim 1, where the one or more characteristics includes scarring of the heart.

3. The system of claim 2, where the one or more characteristics further includes a measurement of the scarring.

4. The system of claim 1, comprising a conduction component that calculates a measure of electrical conduction based on the one or more characteristics of the heart.

5. The system of claim 4, where the level of receptiveness is based on the measure of electrical conduction.

6. The system of claim 1, where the level of receptiveness is further based on a patient demographic.

7. The system of claim 1, where the level of receptiveness is further based on a portion of a patient health history.

8. The system of claim 1, where the level of receptiveness is further based on data derived from a secondary diagnostic test.

9. The system of claim 1, where the heart therapy is an implantable cardiac resynchronization therapy device.

10. A system for optimization of a heart therapy, comprising:
an examination component that identifies at least one characteristic of a heart based at least in part on data derived from single proton emission computed tomography imaging; and
an optimization component that determines a heart therapy device setting based at least in part on the at least one characteristic of the heart.

11. The system of claim 10, comprising a prescription component that determines an individualized implantation procedure based at least in part on the data derived from single proton emission computed tomography.

12. The system of claim 11, where the individualized implantation procedure provides instructions for implanting the heart therapy device in relation to one or more geometric features of the heart.

13. The system of claim 11, where the individualized implantation procedure is further based on an electrical conduction characteristic of the heart.

14. The system of claim 9, where the at least one characteristic of the heart is scarring.

15. The system of claim 9, comprising where the at least one characteristic is further based on data derived from a secondary diagnostic test.

16. A system for modeling a heart, comprising:
a collection component that reads data derived at least in part from a single proton emission computed tomography imaging result; and
a render component that renders a three-dimensional model based on the data.

17. The system of claim 16, comprising a procedure component that displays instructions related to a medical procedure conducted on the heart within the three-dimensional model.

18. The system of claim 16, where the three-dimensional model indicates at least a portion of the model.

19. The system of claim 18, where the portion of the model is scarring.

20. The system of claim 18, where the portion is an implantation location.
